(19) United States
(12) Patent Application Publication
(10) Pub. No.: US 2012/0122115 A1
(43) Pub. Date: May 17, 2012

(54) Bacterial quorum sensing biosensor

(76) Inventors: Richard T. Sayre, Worthington, OH (US); Sathish Rajamani, Columbus, OH (US)

(21) Appl. No.: 11/789,479

(22) Filed: Apr. 25, 2007

Related U.S. Application Data
(60) Provisional application No. 60/794,726, filed on Apr. 25, 2006.

(51) Int. Cl. C07K 19/00; G01N 21/64; C12N 1/21; C12N 15/62; C12N 15/63

(52) U.S. Cl. 435/7.2; 530/350; 536/23.4; 435/320.1; 435/252.33; 436/501; 435/252.31

(57) ABSTRACT

The present invention generally relates to fluorescent resonance energy transfer protein compounds and methods for using such compounds as biosensors. The present invention also relates to one or more nucleic acids for encoding the protein compounds, vectors containing the nucleic acids, cells transformed by the vectors, and methods for making and using the foregoing compositions.

### RELATED APPLICATION DATA

This application claims priority to previously filed U.S. Provisional Application No. 60/794,726, filed on Apr. 25, 2006, entitled “Bacterial Quorum Sensing Biosensor.” The above-identified provisional patent application is hereby incorporated by reference in its entirety.

### FIELD OF THE INVENTION

The present invention generally relates to fluorescent resonance energy transfer protein compounds and methods for using such compounds as biosensors. The present invention also relates to one or more nucleic acids for encoding the protein compounds, vectors containing the nucleic acids, cells transformed by the vectors, and methods for making and using the foregoing compositions.

### BACKGROUND OF THE INVENTION

Many bacterial species produce and monitor low-molecular weight signaling molecules to coordinate gene expression in a population density-dependent manner. This cooperative behavior is known as bacterial quorum sensing (QS), and it controls the expression of a variety of genes. QS signal molecules may regulate a diverse array of functions, including antibiotic production, virulence, bio-film formation, stress and defense responses, motility, metabolism, and activities involved in interactions with eukaryotic hosts. Many bacterial species, including several Vibrio species, use multiple QS signaling pathways. Vibrio harveyi, an opportunistic pathogen of shrimp, is reported to use autoinducer 1 (AI-1), corresponding to N-(3-hydroxybutanoyl)-L-homoserine lactone, for intraspecies signaling and autoinducer 2 (AI-2), (2S,4S)-2-methyl-2,3,3,4-tetrahydroxytetrahydrofuran borate (BAI-2), for interspecies signaling. V. harveyi QS signals regulate gene expression systems involved in bioluminescence, type III secretion, siderophore production, colony morphology, and metalloprotease production.

The AI-2 class of QS signals is derived from 4,5-dihydroxy-2,3-pentanedione (DPD), which is enzymatically synthesized from S-ribosylhomocysteine. BAI-2 is formed from DPD by spontaneous cyclization with borate. In V. harveyi, BAI-2 binds to a periplasmic receptor protein, LuxP, which belongs to a large family of bacterial periplasmic binding proteins (bPBP). Bacteria utilize a variety of bPBPs as signal receptors, chemosensors, and metabolite scavengers. The LuxP class of bPBPs is highly conserved among various Vibrio species which includes several potential human pathogens. The crystal structure of the LuxP protein co-crystallized with its ligand BAI-2 has been determined. Interestingly, BAI-2 had not been identified prior to its structural resolution in the LuxP crystal structure. Recently, structures of the ligand-free form of LuxP, complexed with the periplasmic domain of its signaling partner, LuxQ, have also been determined.

Structure-function analyses of bPBPs in the presence and absence of their respective ligands have demonstrated that the binding of the ligand to its receptor induces substantial conformational changes in the receptor protein. Ligand binding can substantially reduce the protein radius of gyration and the distance between the N- and C-termini of the protein. These ligand-induced structural changes have been exploited for the determination of apparent ligand binding affinities (K_{d}). Ligand-induced conformational changes in bPBPs have been monitored by spectral shifts in environmentally sensitive fluorescent dyes (e.g., acrylodan and fluorescein) that are covalently attached to the receptor protein. Similarly, biosensors that have blue or yellow versions of the jellyfish green fluorescent protein (GFP) translationally fused to the N- and C-termini of the receptor protein have been shown to undergo changes in fluorescence resonance energy transfer (FRET) between the fluorophores upon ligand binding. The resulting FRET-dependent changes in fluorescence emission ratios have been used to calculate apparent K_{d} values.

Notably, U.S. Pat. No. 5,998,204 to Tsien, et al., describes a fluorescent indicator comprising donor and acceptor moieties that are fused to a binding moiety. Furthermore, Tsien, et al. describes such an indicator wherein it changes conformation upon ligand binding thereby resulting a change in fluorescence. However, Tsien, et al. does not teach the use of the LuxP protein, nor does it teach using a fusion protein for detecting quorum sensing compounds. Furthermore, Tsien, et al. does not teach the use of the same donor and acceptor moieties taught herein. Finally Tsien, et al. does not teach a fluorescent indicator that exhibits diminishing FRET upon ligand binding, but rather it describes the reverse.

Currently, bioassays for the detection of BAI-2 take several hours to complete and are subject to substantial environmental and biological perturbations. Thus, there is a need in the art for a more rapid in vitro assay for BAI-2.

### BRIEF DESCRIPTION OF THE FIGURES

FIG. 1 is a drawing showing the operation of biosensors of the present invention;

FIG. 2 is a graph of overlaid fluorescent spectra of CLPY showing a decrease in FRET due to addition of AI-2

FIG. 3(a) is a visual representation of an CLPY fusion protein embodiment of the present invention, wherein the LuxP moiety is not bonded to AI-2;

FIG. 3(b) is a visual representation of the embodiment of FIG. 1, wherein the LuxP moiety is bonded to AI-2;

FIG. 4 is a multiple sequence alignment of LuxP protein sequences found in V. harveyi (ViHar), V. parahaemolyticus (ViPar), V. vulnificus (ViVul), V. cholerae (Vicho), V. anguillarum (ViAng), and V. fisheri (ViFis);

FIG. 5(a) is a drawing of a fusion protein of the present invention showing three possible FRET effects due to ligand binding;

FIG. 6(a) is a graph illustrating spectra relating to an increase in FRET characterized by a slow-time dependent change;

FIG. 6(b) is a graph illustrating a spectrum relating to a constant FRET;

FIG. 6(c) is a graph illustrating molar ellipticity versus time at 220 nms;

FIG. 6(d) illustrates amino acid deletion from the linker region between CFP-LuxP-YFP fusion (CLPY to RYSDN) to various LuxP biosensors;

FIG. 6(e) is a graph illustrating intensity versus time for CFP and YFP in CLPY protein monitored for fluorescence yield and time-dependent YFP fluorescence yield increase due to YFP maturation;

FIG. 8 is a diagram of the pQE-30 vector;

FIG. 9 contains graphs illustrating plots of emission intensity versus wavelength for FRET responses of dimeric (91 to 99 mL) and monomeric (106 to 114 mL) CLPY to buffer (top lines) and 1.5 μM BAI-2 (bottom lines);

FIGS. 10(a) through (c) are graphs illustrating: (a) FRET response of CLPY to different concentrations of BAI-2 in borate-free buffer (), DPD (⋄), and borate (♦), where the error bars represent the standard deviations calculated from the FRET assays from three independent experiments, (b) response of LuxP M2CLPY to different concentrations of BAI-2, and (c) response of LuxP M3CLPY to different concentrations of BAI-2;

FIGS. 12(a) and (b) are graphs illustrating the quantification of BAI-2 from V. harveyi cultures using the CLPY biosensor, where FIG. 12(a) is a graph illustrating wild-type V. harveyi BAI-2 levels monitored as a function of time and culture density, and FIG. 12(b) is a graph illustrating the response of CLPY fluorescence to culture filtrate from wild-type (BB120) V. harveyi (▴) and LuxS⁻ mutant (MM32, Δ) mid- to late-log phase culture filtrate, where the error bars represent the standard deviations from three independent experiments;

FIG. 13(a) is a set of overlaid fluorescence spectra showing 0.015 mgs/mL M3CLPY response to V. harveyi BB120 cell-free supernatant (250 μl) for 16 hours at 28° C.;

FIG. 13(b) is a bar graph showing the amount of FRET reduction from M3CLPY after 2.5 hours incubation with 16 hours old strains of V. harveyi culture filtrates (trials were carried out with 3 independent cultures, with fresh AB media as control);

FIG. 13(c) is a bar graph showing FRET ratio versus strains of 4.5 hours old (2% innoculum) MM30 and MM32 strains monitored with addition of borate containing buffer or 100 μM DPD:Borate (1:4) and growing for additional 11.5 hours where the FRET measurement data is confirmed in FIG. 13 (d);

FIG. 13(d) is an immunoblot (anti-GFP antiobody);

FIG. 14 is a pair of time based fluorescence curves showing the fluorescence yields of M3CLPY donor and acceptor in the presence of cell-free filtrate from V. harveyi BB120 cell culture; and

FIG. 15 is a photograph of a Western blot wherein M3CLPY is incubated with various V. harveyi strains having QS pathway mutants.

### SUMMARY OF THE INVENTION

The present invention generally relates to fluorescent resonance energy transfer protein compounds and methods for using such compounds as biosensors. The present invention also relates to one or more nucleic acids for encoding the protein compounds, vectors containing the nucleic acids, cells transformed by the vectors, and methods for making and using the foregoing compositions.

In one embodiment, the present invention relates to quorum sensing compound biosensors. More particularly, the present invention relates to biosensors for quantitatively detecting the concentration of autoinducer-2 (AI-2) quorum sensing compounds using fluorescent resonant energy transfer (FRET).

In another embodiment the present invention also relates to a fusion protein comprising SEQ ID 8. The present invention also relates to a fusion protein comprising a LuxP moiety, wherein the LuxP moiety is disposed between a donor and acceptor moiety; a cyan fluorescent protein donor moiety connected to the LuxP moiety; and a yellow fluorescent protein acceptor moiety connected to the LuxP moiety, wherein the yellow fluorescent protein is adapted to be substantially insensitive to chloride ion concentration, and wherein the donor and acceptor moieties disposed so that they are capable of fluorescent energy transfer when no ligand is bound to the LuxP moiety, and exhibit diminished or no fluorescent energy transfer when ligand binds to LuxP.

In another embodiment the present invention further relates to an isolated nucleic acid coding for a fusion protein comprising SEQ ID 7. The present invention also relates to an expression vector containing SEQ ID 7. Furthermore, the present invention relates to a host cell transformed by an expression vector containing SEQ ID 7. The present invention also relates to method for detecting autoinducer-2, comprising providing a solution of the fusion protein set forth above; obtaining a first fluorescence measurement of the solution; adding a portion of a cell-free lysate to the solution, wherein the lysate can contain a ligand capable of binding to the fusion protein; obtaining a second fluorescence measurement of the solution after adding the portion of cell-free lysate; calculating a degree of fluorescent energy transfer by comparing the second measurement to the first measurement, and correlating the degree of fluorescent energy transfer to a ligand concentration.

In still another embodiment, the present invention relates to a method for detecting protease activity, comprising providing a solution of the fusion protein of set forth above, wherein the fusion protein further comprises one or more peptide sequences fused to the donor, the acceptor or both the donor and the acceptor, wherein the one or more sequences attenuate fluorescent energy transfer between the donor and the acceptor as a function of protease activity; obtaining a first fluorescence measurement of the solution; adding a portion of a cell-free lysate to the solution, wherein the lysate can contain a ligand capable of binding to the fusion protein, and can contain one or more proteases; obtaining a second fluorescence measurement of the solution after adding the portion of cell-free lysate; calculating a degree of fluorescent energy transfer by comparing the second measurement to the first measurement; correlating the degree of fluorescent energy transfer to a ligand concentration; and correlating the ligand concentration to protease activity.

### DETAILED DESCRIPTION OF THE INVENTION

The present invention generally relates to fluorescent resonance energy transfer protein compounds and methods for using such compounds as biosensors. The present invention also relates to one or more nucleic acids for encoding the protein compounds, vectors containing the nucleic acids, cells transformed by the vectors, and methods for making and using the foregoing compositions.

As is noted above, currently bioassays for the detection of BAI-2 take several hours to complete and are subject to substantial environmental and biological perturbations. Thus, a more rapid in vitro assay for BAI-2 would be an attractive alternative. In one embodiment of the present invention a highly sensitive, FRET-based BAI-2 biosensor is disclosed. In another embodiment, the FRET-based BAI-2 biosensor is used to quantify BAI-2 levels in V. harveyi cell culture filtrates and to demonstrate that BAI-2 levels increase rapidly as the cell density increases—followed by a decrease in BAI-2 levels before the cells reach a stationary phase. While not wishing to be bound to any one use, or set of uses, the BAI-2 biosensor of the present invention has potential applications in monitoring bacterial population densities, including potential human pathogens.

As used herein, the lowercase letter “m” preceding a protein compound name indicates the monomeric form of the protein. However, protein compounds names lacking the “m” modifier can also encompass and/or indicate a monomeric form. For example, the name CLPY is a general term describing a fusion protein of the present invention, and CLPY specifically indicates the monomeric form thereof. However, CLPY alone and without the “m” modifier encompasses all forms of the fusion protein including the monomeric form. The same is true for the YFP and CFP proteins as well as others, as will be clear from context.

In one embodiment, the present invention generally relates to a method for detecting bacterial quorum sensing compounds or mimics thereof, and biosensors embodying such method. In general one method of the present invention involves binding a quorum sensing compound to a fluorescent protein complex, which results in a conformational change in the complex and thereby causes a characteristic change in resonance energy transfer.

The formula below illustrates a boron derivative of autoinducer 2 (BAI-2):

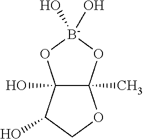

A sensing compound within the scope of the present invention includes a three-protein complex comprising: (1) a protein capable of binding to the class of quorum sensing (QS) compounds known as autoinducers, such as autoinducer 2 (AI-2) and/or derivatives thereof; (2) a modified form of cyan fluorescent protein (CFP); and (3) a modified form of yellow fluorescent protein (YFP). The QS binding protein is located between the CFP and YFP moieties, and holds them in a manner that enables fluorescent resonance energy transfer (FRET).

Optionally, the biosensing fusion protein can include linkers that tether the CFP and YFP moieties to the QS-binding moiety. In some embodiments, such linkages can be useful for positioning the CFP and YFP moieties so that their wave functions sufficiently overlap. Typically, useful linkages can comprise relatively flexible and sterically unhindered moieties, such as glycine, alanine and polymers or copolymers thereof. Alternatively, other embodiments can include relatively inflexible linking moieties, such as amino acids having bulky side chains, e.g. phenylalanine, tyrosine, etc. Still further embodiments can comprise combinations of flexible and inflexible linking moieties, thereby achieving an intermediate degree of flexibility.

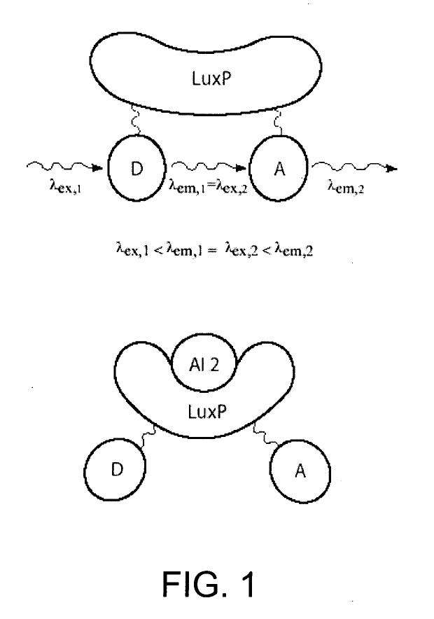

In general, FRET occurs when the emission spectrum of a donor moiety overlaps with the absorption spectrum of an acceptor moiety, and the donor and acceptor are close enough to electronically couple. A drawing of an example arrangement can be seen in FIG. 1. Preferably, the absorption spectra of the donor and acceptor are well separated, so that the wavelength selected for exciting the donor (λ_{ex,1}) does not also excite the acceptor (λ_{ex,1}≠λ_{ex,2}). If the absorption spectra of the donor and acceptor appreciably overlap at λ_{ex,1} it would interfere with distinguishing between acceptor excitation due to energy transfer versus direct irradiation.

The efficiency of FRET depends on several factors including wave function overlap of the donor and acceptor. This, in turn, depends on the distance between the donor and acceptor, as well as their relative orientation. Additionally, FRET efficiency depends on the quantum efficiency of the donor, i.e. the ratio of excitation energy absorbed versus emitted. FRET efficiency can be quantitatively described according to the Forster equation:

$$E = \frac{\vert {F}_{0} - F \vert}{{F}_{0}} \qquad Equation 1$$

In this equation, E is efficiency, F₀ is the fluorescent intensity of the donor in the absence of the acceptor, and F is the fluorescent intensity of the donor in the presence of the acceptor.

A typical arrangement for FRET measurements includes the following. A broad band light source, such as a mercury arc lamp, is used to generate white light. The white light is passed through a monochromator so as to select a particular wavelength λ_{ex,1} for exciting the donor. As noted above, λ_{ex,1} should excite substantially only the donor, and not the acceptor. The selected wavelength is then directed to a sample containing the biosensor, wherein the donor absorbs a portion of the incident λ_{ex,1} light. The donor, in turn, emits a portion of the absorbed light at a longer wavelength λ_{ex,1}, which falls within the absorption band of the acceptor. Thus, λ_{em,1}=λ_{ex,2} and the acceptor absorbs a portion of the donor's emitted light.

One of ordinary skill in the art will readily recognize that molecular emission spectra comprise a band of wavelengths rather than a single wavelength. Thus, λ_{em,1} represents a range of wavelengths and/or any point within such range. Similarly, one of ordinary skill in the art will readily recognize that molecular absorption spectra comprise bands of wavelengths rather than a single wavelength. Accordingly, λ_{ex,2} represents a range of wavelengths and/or any point within the range, wherein such wavelength is found in the emission spectrum of the donor and the absorption spectrum of the acceptor.

Additionally, a portion of the donor's excited state energy is transferred directly to the acceptor via a FRET process, bypassing donor emission. Thus, the acceptor enters an excited state, relaxes, and emits a still longer range of wavelengths, λ_{em,2}. Finally, a representative sample of the total emission from both the donor and acceptor is collected and passed through a second monochromator in slew mode, thereby generating an emission spectrum. Such emission spectrum comprises a composite of donor and acceptor emission.

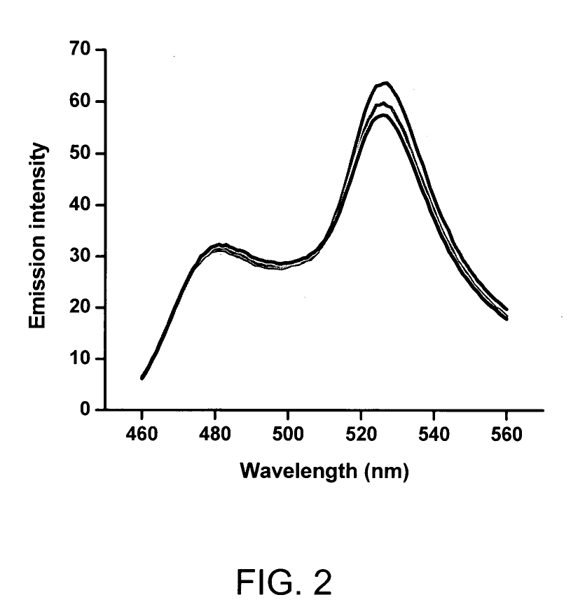

A typical steady state fluorescent emission spectrum of a molecule exhibiting FRET is shown in FIG. 2, which comprises two peaks. FIG. 2 illustrates a response of CLPY to unsaturating [1.0 nM (top line], partially saturating [285 nM (middle line)], and fully saturating [5.6 μM (bottom line)] concentrations of BAI-2 ligand. The shorter wavelength peak is donor emission and the longer wavelength peak is acceptor emission. Notably, a decrease in shorter wavelength emission intensity corresponds to an increase in longer wavelength emission intensity, which is due to energy transfer from the donor to the acceptor. One way to quantify energy transfer using this data is to take the ratio of the acceptor emission maxima with and without quorum sensing ligand present. For instance, if the acceptor's maximum fluorescence intensity is I₁ in the presence of quorum sensing ligand, and I₂ in the absence of quorum sensing ligand then FRET can be represented as

$$FRET = \frac{{I}_{A , 1}}{{I}_{A , 2}} \qquad Equation 2$$

According to this equation, FRET equals 1 when no energy transfer occurs, and decreases as ligand concentration increases. Since energy transfer is maximal when no quorum sensing compound is present, FRET<1 indicates that energy transfer is occurring. This method assumes constant fluorophore concentration.

Another way of quantifying FRET, comprises taking the ratio of the donor and acceptor emission intensities of each sample.

$${FRET}_{1} = \frac{{I}_{D , 1}}{{I}_{A , 1}} , {FRET}_{2} = \frac{{I}_{D , 2}}{{I}_{A , 2}} , … {FRET}_{n} = \frac{{I}_{D , n}}{{I}_{A , n}}$$

In one embodiment, the sample having no quorum sensing ligand is designated as the baseline, e.g. FRET₁. Each of the samples is therefore normalized to the baseline sample. Thus, changes in FRET are judged in terms of the ratio of FRETₙ to FRET₁:

$${FRET}_{normalized} = \frac{{FRET}_{n}}{{FRET}_{1}} \qquad Equation 3$$

In addition to the foregoing, FRET can be quantified by time-resolved fluorescence spectroscopy. Usually, the appropriate time-scale for such measurements falls in the nanosecond regime; however, others may fall in the pico or femtosecond regimes. In any case, an increase in FRET is indicated by a reduction in donor excited state life time relative to an appropriate control sample. As will be apparent to one of ordinary skill in the art, there are a variety of alternative methods for quantifying FRET. Thus, these are also within the scope of the present invention.

In one embodiment, the biosensor of the present invention comprises at least the following three components: (1) a receptor protein such as LuxP; (2) a donor such as a variant of the Cyan Fluorescent Protein (CFP); and (3) an acceptor such as a variant of the Yellow Fluorescent Protein (YFP). More particularly, the LuxP protein is bound to the CFP and YFP components so that it holds CFP and YFP in close proximity. This embodiment can also include one or more linkers that serve to tether CFP and YFP to LuxP. Still more particularly, LuxP holds CFP and YFP in close enough proximity for them to experience fluorescent resonant energy transfer (FRET). When the LuxP protein binds with an AI-2, it results in a conformational change wherein the CFP and YFP move farther apart and experience less FRET, or no FRET. Thus, the amount of AI-2 can be quantitatively determined as a function of energy transfer. Alternatively, fusion proteins of the present invention can be used to qualitatively determine the presence or absence of quorum sensing compounds, such as one of the AI-2 class.

FIG. 2 shows the effect of AI-2 on the fluorescent spectrum of CLPY. The spectrum comprises two primary peaks: one at about 480 nm, and a second at about 528 nm. The 480 nm emission is attributed to CFP, and the 528 nm emission is attributed to YFP. When the CFP and YFP moieties are in sufficiently close proximity YFP absorbs a portion of the CFP moiety's excited state energy. Accordingly, the emission of YFP increases due to FRET, and that of CFP diminishes. Thus, the concentration of AI-2 can be quantitatively determined as a function of the ratio of yellow to blue emission intensities. In general, this same theory of operation holds for each embodiment of the present invention.

Again referring to FIG. 2, the spectrum having a much greater yellow emission than blue results from the sample having no AI-2. The spectrum having roughly equal yellow and blue emissions results from the sample containing AI-2. The change in the ratio of yellow to blue emission intensities is due to an AI-2 concentration dependant reduction in FRET. When no AI-2 is present, substantially all CLPY molecules engage in FRET. However, when AI-2 is introduced it binds to CLPY causing a conformational change and a consequent reduction in FRET. Thus, a higher yellow to blue intensity ratio indicates a lower amount of AI-2.

According to well known methods, the foregoing concentration dependant FRET effect can be used to determine the amount of AI-2 ligand, or other analyte. For instance, a calibration curve can be constructed by running a series of samples containing known amounts of AI-2. Unknown concentrations can then be determined by comparison to the calibration curve.

As noted above, the CLPY embodiment comprises CFP and YFP moieties. Both CFP and YFP are mutants of wild-type Green Fluorescent Protein (GFP), which exhibits spectral artifacts due to pH, temperature, and in some cases chloride ion concentration. In contrast CFP and YFP do not exhibit such artifacts; their fluorescence spectra are substantially independent of pH and temperature over a useful range. Furthermore, some embodiments include a mutant form of YFP that is substantially insensitive to chloride ion over a useful range. For example, one such mutant can be made according to the following reference, which is hereby incorporated by reference in its entirety: Nagai, T. et al., A variant of yellow fluorescent protein with fast and efficient maturation for cell-biological applications, Nat. Biotechnol. 20, 87 to 90 (2002).

The sequence of monomeric CFP (mCFP) is set forth in SEQ ID 8, residues 1 through 254. The mutations of GFP that result in CFP include each of the following: tyrosine 66 to tryptophan (Y66W), asparagine 146 to isoleucine (N146I), methionine 153 to threonine (M153T), valine 163 to alanine (V163A), and luecine 221 to lysine (L221K). The L221K mutation prevents dimerization. The quantum yield of mCFP lacking the L221K mutation is 0.42.

The sequence of monomeric YFP (mYFP) is set forth in SEQ ID 8, residues 606 through 841. The mutations of GFP that result in YFP include each of the following: serine 65 to glycine (S65G), valine 68 to leucine (V68L), glutamine 69 to lysine (Q69K), serine 72 to alanine (S72A), threonine 203 tyrosine (T203Y), and leucine 221 to lysine (L221K). The L221K mutation prevents dimerization. The quantum yield of mYFP lacking the L221K mutation is 0.67.

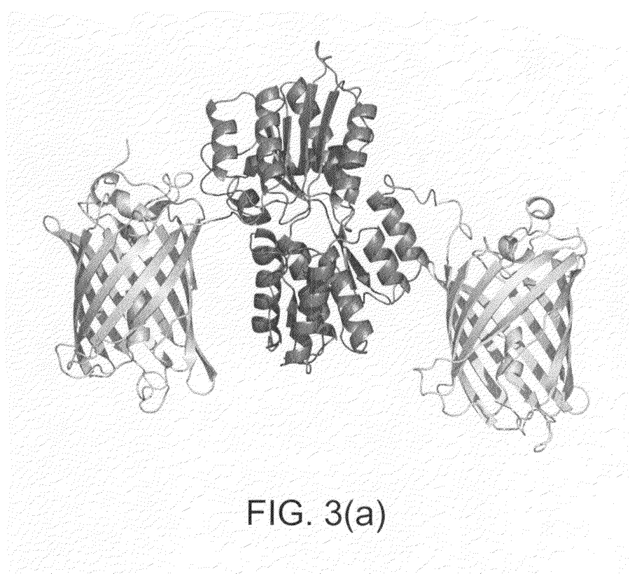

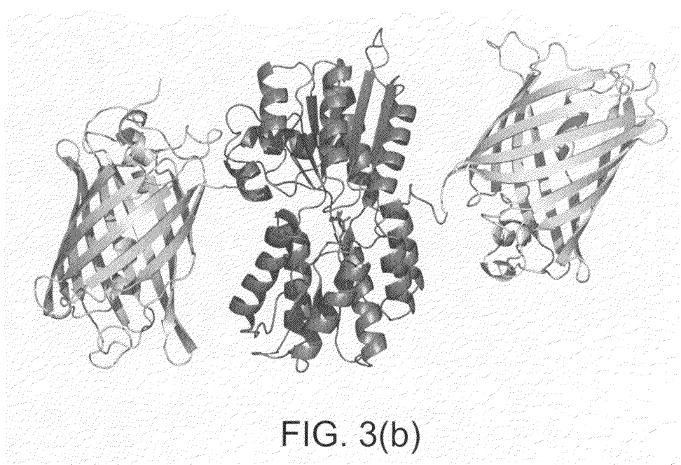

A fusion protein made from CFP, LuxP, and YFP is shown in FIG. 3, and the sequence is set forth in SEQ ID 8, residues 1 through 841. Such fusion proteins can also include a variety of linking sequences between mCFP, LuxP, and mYFP. For instance, Table 1 below 6 sets forth several linking sequences within the scope of the present invention.

TABLE 1

| Cyan | Linker-1 | Binding Moiety | Linker-2 | Yellow |
|---|---|---|---|---|
| mCFP | TADG | VLNG LuxP RYSDN | GGAAA | mYFP |
| mCFP | TADG | LuxP RYSDN | GGAAA | mYFP |
| mCFP | TADG | LuxP RYSDN | G | mYFP |
| mCFP | G | LuxP RYSDN | G | mYFP |
| mCFP | G | LuxP | G | mYFP |

However, these are only illustrative examples, and not an exclusive list. Moreover, some embodiments of the present invention can also lack one or both linking sequences. Note that the binding moiety comprises LuxP or any of a variety of deletion mutants. For instance, in the first line of Table 1 the binding moiety comprises LuxP including the VLNG residues, which are also part of the LuxP moiety. In the second, third, fourth and fifth lines the binding moiety comprises deletion mutants of LuxP, wherein the VLNG sequence is deleted. Additionally, line five comprises a LuxP deletion mutant also lacking the RYSDN residues.

### Construction of LuxP Based FRET Biosensor:

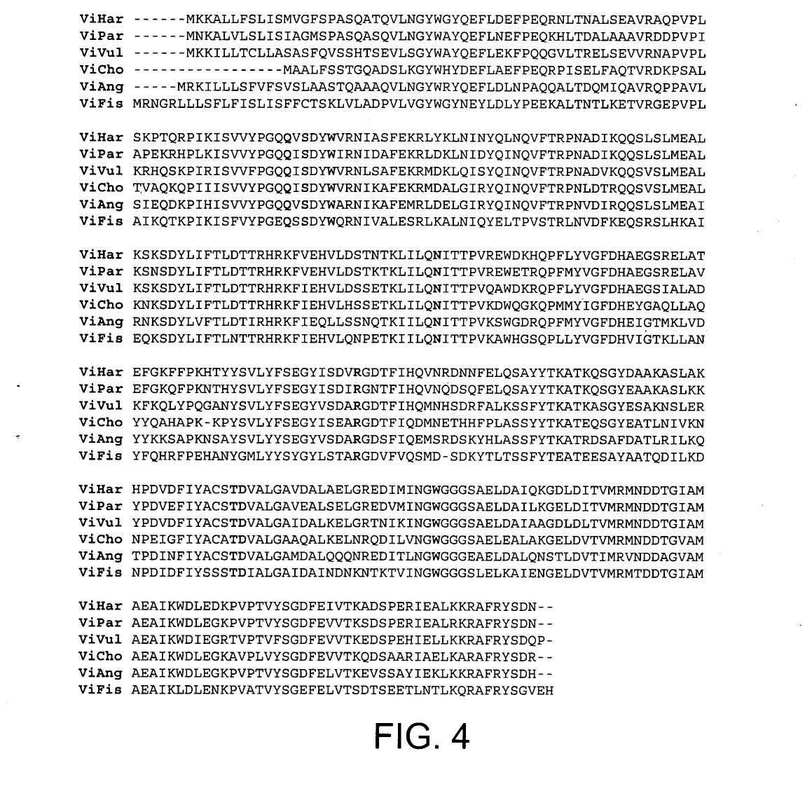

In one embodiment the biosensing fusion protein of the present invention is constructed according to the following. LuxP protein is conserved in several Vibrio species. A BLASTP search (www.ncbi.nlm.nih.gov/BLAST/) using the V. harveyi LuxP protein revealed the presence of LuxP in a variety of related organisms, such as V. harveyi, V. parahemolyticus, V. vulnificus V. cholrae, and V. anguillarum. Multiple sequence alignment of LuxP sequences reveals a highly conserved amino acid sequences and BAI-2 binding residues (FIG. 4).

Generally, organisms with highly conserved LuxP proteins also have similarly conserved luxS genes, which are involved production of DPD (a precursor of AI-2). Several of these organisms are known to use BAI-2 mediated QS gene regulation. In general, LuxP ligand recognition results in a conformational change. The reason for this is that proteins such as LuxP, commonly have their ligand binding site between two lobes that are connected by a flexible hinge.

FRET can be used to study these conformational changes because it is useful over distances spanning between about 10 to 100 Angstroms. In one embodiment, V. harveyi LuxP devoid of signal peptide is fused to monomeric forms of GFP variants cyan fluorescent protein (mCFP) and yellow fluorescent protein (mYFP). The relative distance between the N- and C-termini of LuxP is about 37 Angstroms when it is bound to the BAI-2 ligand. Therefore, FRET effects should be observable.

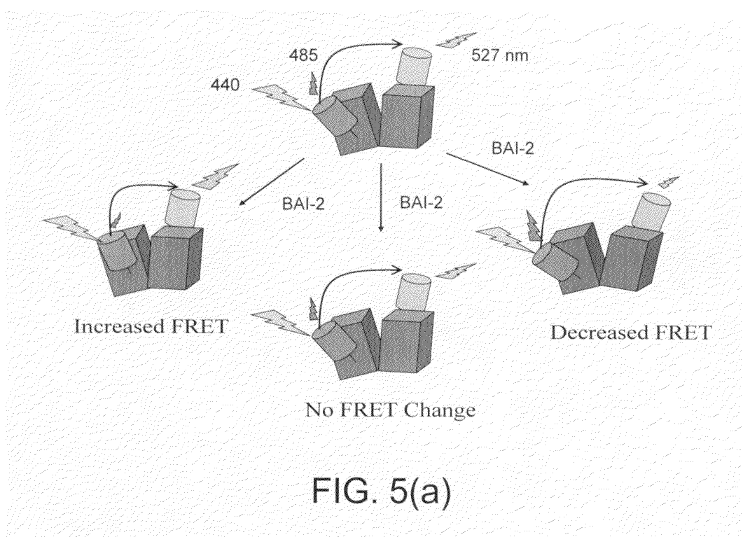

The positioning of the N- and C-termini of the protein is not consistent with earlier models of similar FRET sensors. Particularly, a lateral termini positioning in the LuxP binding site complicates structure-based predictions of FRET signal modulation. Therefore, in FIG. 5(a) several illustrative possible scenarios for BAI-2 induced CLPY biosensor signal changes, not being bound by any particular theory. With regard to FIG. 5(a), this Figure illustrates possible mechanisms for ligand-induced FRET changes in CLPY. In FIG. 5(a) the CLPY protein is represented as a CFP (forward cylindrical objects), LuxP (rectangular objects), YFP (back cylindrical objects) fusion protein. In the absence of BAI-2, CFP exhibits high fluorescence. There are three possible outcomes following ligand binding (a) Increased FRET; (b) No FRET Change; and (c) Decreased FRET associated with either a conformational-induced change in the distance between CFP and YFP, a change in the orientation of the chromophores, or no change at all (b).

### Ligand-Independent FRET:

Some embodiments of the present invention can exhibit ligand concentration-independent FRET modulations. Thus, one aspect of the present invention is directed to elucidating the cause of such effect and applying this knowledge to create embodiments that are less dependant upon or free from concentration-independent FRET changes.

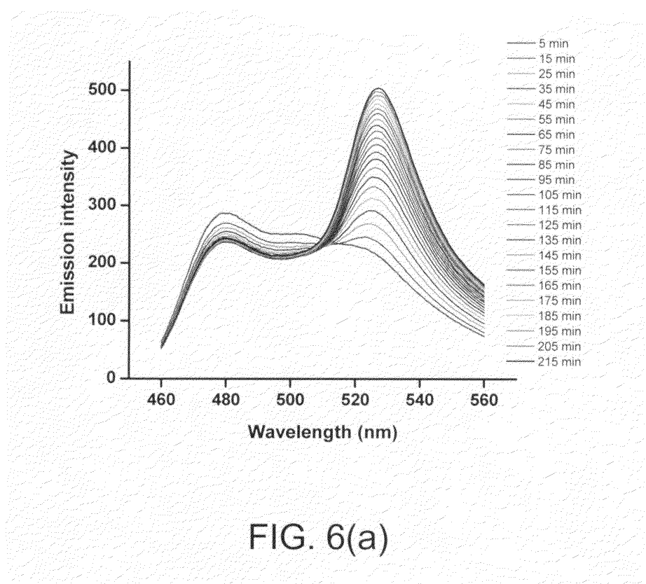

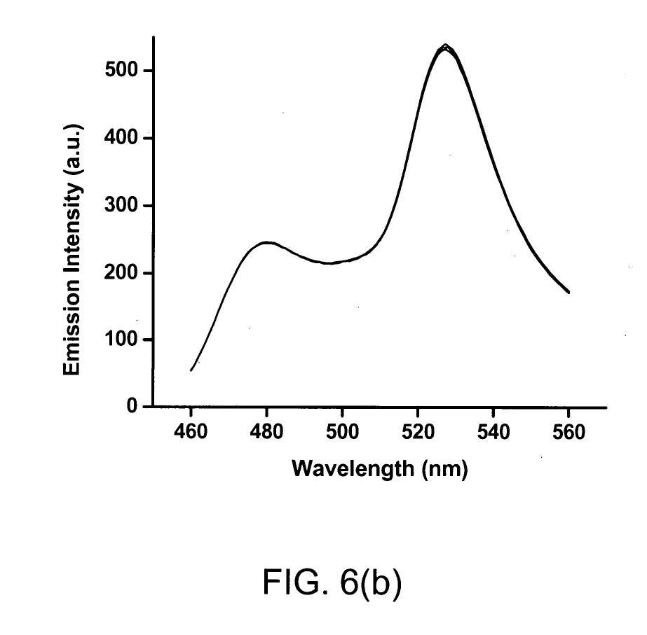

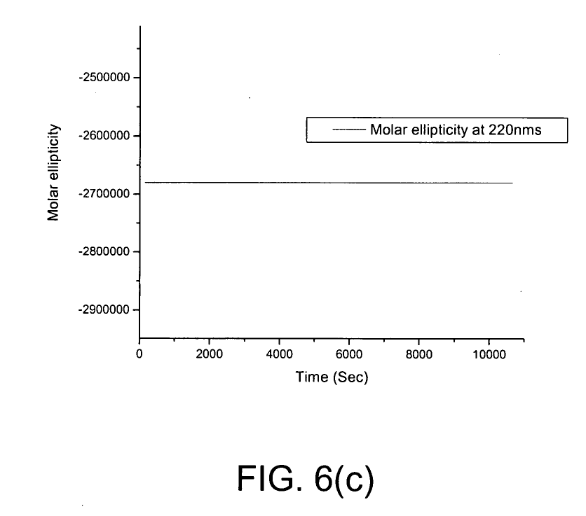

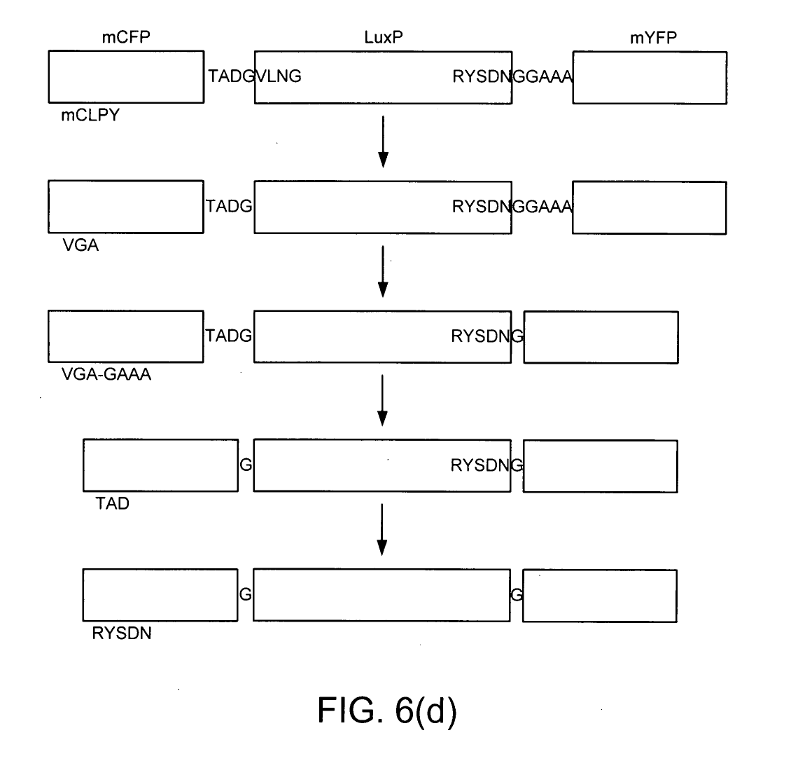

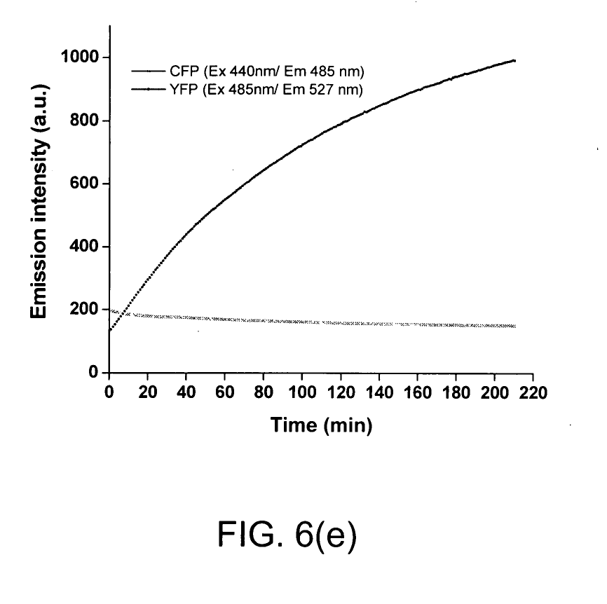

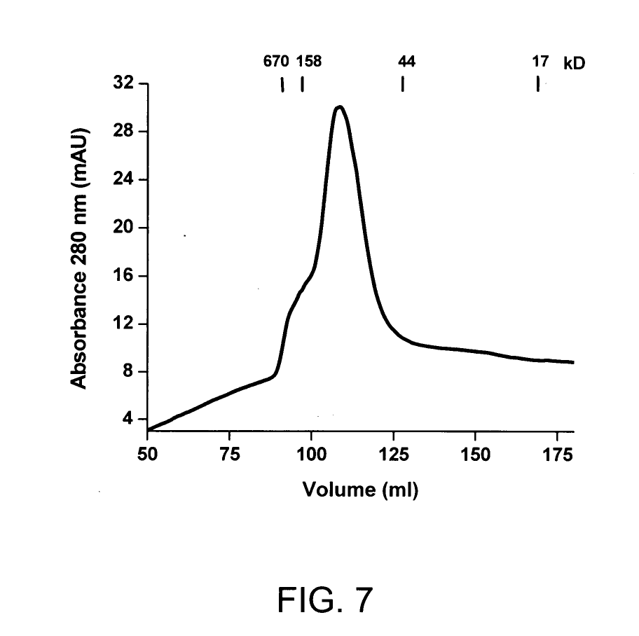

In one embodiment, a LuxS⁻ strain of BL21 E. coli is used to express the CLPY fusion protein. When CLPY is spectrofluorometrically analyzed immediately after purification, it exhibits a ligand-independent increase in FRET. This increase in FRET is characterized by a slow time-dependent change as shown in FIG. 6(a).

Room temperature fluorescence measurements show that FRET efficiency changes over the course of nearly five hours. Eventually, it stabilizes and the signal becomes a constant with respect to time. At that point the equilibrated protein has a constant FRET ratio of 2:1 (527 nm/485 nm) as shown in FIG. 6(b). The rate of CLPY protein maturation is slower at lower temperatures. For instance, at 4° C. CLPY needs about 48 hours to equilibrate, which demonstrates a temperature dependency on CLPY maturation.

Long time-scale protein folding processes are ruled out as the cause of the concentration independent FRET effect. Circular dichroism (CD) is monitored at 220 nm. Purified CLPY is monitored at room temperature for 3.5 hours and showed no detectable CD signal change (FIG. 6(c)). However, simultaneous fluorescence measurements show a concentration-independent increase in FRET as a function of time. Therefore, secondary structural changes are ruled out as the cause of the FRET effect.

Next, the linking sequences are ruled out as the source of the concentration independent FRET effect. A set of CLPY mutants are prepared, which differ only in the sequence of the linkers. Specifically, the mutants comprise sequence deletions, as shown in FIG. 6(d). Similar to CLPY, each of the mutants demonstrates the same concentration independent FRET effect as a function of time. Therefore, the varied portions of the sequences are ruled out as the source of the FRET effect. Without being bound by any one theory, it is believed that the time-dependant FRET phenomenon is the result of slow maturation of YFP as compared to CFP. This accounts for the observed increase in FRET.

Accordingly, the present invention includes a biosensing fusion protein, wherein the protein is capable of producing a signal that is substantially time independent. Furthermore, time independence can be achieved in any of a wide variety of ways including, without limitation, aging, temperature treatment, sonication, absorption of electromagnetic radiation (e.g., infrared or microwave), and any combination thereof.

Mature CLPY responds to additions of BAI-2 with decreased FRET efficiency as shown in FIG. 2, and is characterized by an isosbestic point at 511 nm. Recently, it was reported that ligand-free LuxP at concentrations around 200 μM can exist as mixtures of monomeric and dimeric forms. However, upon ligand binding, LuxP predominantly occupies the monomeric form. This implies the CLPY FRET change could also be caused by ligand induced dimer to monomer LuxP switch.

GFPs are known to dimerize with a K_{d} of about 100 μM. However, dimerization can be essentially eliminated by incorporating L221K mutation, which renders them monomeric regardless of concentration (i.e., mCFP and mYFP). After purification the mature CLPY sample is concentrated to 20 μM (2.5 mg/mL) and analyzed by size exclusion chromatography (SEC) as shown in FIG. 7, the CLPY protein is detected at 280 nm and is found to exist predominantly in its monomeric state (98 kDa, FIG. 7). Subsequent FRET measurements using monomeric protein fractions (volumes 106-114 mL, 0.015 mg/mL) demonstrate a similar ligand-induced FRET change recorded earlier (FIG. 2). According to these results no dimeric CLPY state can be detected. Thus, the LuxP biosensor functions as a monomer.

However, LuxP dimerization is a highly concentration dependent process. Furthermore, the CLPY FRET response to BAI-2 is observed at protein concentrations about 175 times lower than that which is used in SEC runs (i.e., 150 nM versus 25 μM). The dimeric form should not exist at such low concentrations. Therefore, an intermolecular FRET mechanism can therefore be ruled out.

### Experimental Procedures:

Bacterial Strains and Media:

Escherichia coli BL21 (luxS⁻) and V. harveyi strains BB120 (wild-type), MM30 (luxS⁻), and MM32 (luxS⁻, luxN⁻) are available from The American Type Culture Collection (ATCC) or from Bonnie L. Bassler (Princeton University, Princeton, N.J.). Luria-Bertani (LB) medium is used to grow E. coli. Luria-Marine (LM) medium, autoinducer bioassay (AB) medium, or boron-free AB medium are also used for the growth of V. harveyi strains. As needed, antibiotics such as ampicillin (100 mg/L) and kanamycin (100 mg/L) are used with growth media. For solid media, 15 g/L select agar (Invitrogen) is used with the liquid media. V. harveyi cell numbers are counted by plating serial dilutions of cultures on LM medium.

Site-Directed Mutagenesis and Cloning:

Site-directed mutations were carried out using the QuikChange mutagenesis kit (Stratagene). The perGFP plasmid containing the gene encoding the green fluorescent protein (GFP) is used for creating cyan and yellow variants CFP and YFP, respectively. An L221K mutation is introduced into the gene encoding CFP or YFP to reduce the possible level of intermolecular dimerization. Cloning vector pSP72 (Promega) is used for creating 5′ to 3′ end-to-end gene fusions of PCR fragments containing the genes encoding CFP (5′ KpnI/3′ EagI), V. harveyi LuxP (70 to 1095 bp; 5′ EagI/3′ NotI), and YFP (5′ NotI/3′ PstI), generating plasmid pSP72-CLPY. The CLPY gene fusion is removed using the restriction enzymes KpnI and PstI and subcloned into a pQE-30 bacterial expression vector (Qiagen) containing an N-terminal six-His tag, generating plasmid pQE30-CLPY. LuxP BAI-2 binding site mutants pQE30-M2CLPY (Q77A and S79A) and pQE30-M3CLPY (Q77A, S79A, and W82F) are created using pQE30-CLPY as a template and confirmed by DNA sequence analysis.

Protein Overexpression, Purification, and Characterization:

BL21 (luxS⁻) cells transformed with pQE30-CLPY or LuxP mutant constructs are grown at 28° C. in LB broth supplemented with 100 mg/L ampicillin. Protein expression is induced with 0.3 mM isopropyl thiogalactoside at an optical density (OD₆₀₀) of 0.6 and grown for an additional 6 hours. Bacterial cells are harvested by centrifugation, and further purifications were carried out at 4° C. Cells are re-suspended in 25 mM NaH₂PO₄—Na₂HPO₄ (pH 8.0), 35 mM NaCl, and 10 mM imidazole (buffer A) and sonicated with 15 mM 2-mercaptoethanol and 1 mM phenylmethanesulfonyl fluoride. Clarified cell lysate is then loaded onto a His-Select-HC nickel affinity gel (Sigma) equilibrated with buffer A. The protein-bound resin is washed with buffer A and eluted with 25 mM NaH₂PO₄—Na₂HPO₄ (pH 8.0), 35 mM NaCl, and 50 mM imidazole (buffer B). The protein purity is judged by SDS-PAGE, and the protein yield is determined using a Bradford protein assay kit (Bio-Rad). Fluorescence measurements are carried out at room temperature using a Cary Eclipse spectrofluorometer (Varian Inc.) set in a scanning mode. Wild-type and mutant forms of CLPY are monitored on the column by CFP fluorescence emission (λₑₓ=440 nm, 5 nm slit; λₑₓ=460 to 560 nm, 5 nm slit). Separation of CLPY monomers and dimers is carried out by size-exclusion chromatography using a FPLC system fitted with a Sephacryl S-100 HR 26/60 column (Amersham Biosciences). The column is equilibrated with buffer B, and 500 μL of 2.5 mg/mL CLPY protein is applied at a flow rate of 0.8 mL/min. Protein standards [thyroglobulin (670 kDa), IgG (158 kDa), ovalbumin (44 kDa), and myoglobin (17 kDa)](Bio-Rad) are run under similar conditions.

Synthesis of a Boron Derivative of Autoinducer 2 (BAI-2):

Nucleosidase Pfs and Co²⁺-substituted S-ribosylhomocysteinase (LuxS), both from Bacillus subtilis, are overexpressed in E. coli BL21(DE3) and purified to apparent homogeneity. S-Adenosylhomocysteine (SAH), 5,5′-dithiobis(2-nitrobenzoic acid) (DTNB), and all other chemicals and reagents are purchased from Sigma-Aldrich. SAH (15 mM) is incubated with nucleosidase Pfs (4 μM) for 6 hours at room temperature to give S-ribosylhomocysteine (SRH), and the completion of the reaction is monitored spectrophotometrically on the basis of the absorption difference between SAH and adenine (Δε₂₇₆=−1.4 mM⁻¹cm⁻¹). The crude SRH is directly used in LuxS reactions without further treatment. BAI-2 is prepared by incubating SRH (3 mM) and LuxS (2.0-2.5 mg/mL) at room temperature overnight in 100 mM NaH₂PO₄—Na₂HPO₄ (pH 7.5), 20 mg/mL NaHCO₃, and 12 mM borate (4 equivalents). The DPD yield is determined indirectly by assaying the amount of homocysteine released using DTNB, and the concentration of BAI-2 is corrected by a factor of 0.1 on the basis of the ¹¹B NMR analyses. DPD is synthesized as described above without the addition of borate in the LuxS reaction buffer.

Cell-Free Culture Filtrate Preparation:

V. harveyi is cultured with 2% inoculum of saturated starter culture, and grown at about 28° C. for about 15 hours. Cell supernatant is collected by centrifuging the culture in a bench top centrifuge (Ependorff 5415 D) at about 16,100 g for 1 minute. The cell supernatant is transferred to a 3K Microsep™ centrifugal device (Pall Life Science), which is washed prior with sterile double distilled water. Cell supernatant (2.0 mL) is centrifuged (Sorvall RC5C) at 5000 g for 30 minutes at 4° C. The protease-free filtrate is used to make various dilutions in fresh borate-free AB media and for BAI-2 quantification. For monitoring extracellular protease activity the cell supernatant from 16 hours old culture is passed through HT Tuffryn® Acrodisc® syringe filter (Pall Life Sciences). A 250 μL portion of this cell-free filtrate is mixed with M3CLPY and used for assays. For extracellular protease recovery study, 250 μL of cell-free filtrate is used with 1.0 mL of 0.015 mg/mL protein.

Binding Affinity Determination:

The YFP or CFP fluorescence response of wild-type and mutant forms of CLPY (1.0 mL, 0.015 mg/mL) to different ligands is measured 5 minutes after addition of the ligand to the protein at room temperature. No time-dependent changes in the CLPY FRET ratio are observed after incubation for 5 minutes. All BAI-2 dilutions are carried out in a borate-free buffer to maintain the DPD:borate ratio (1:4). The CLPY FRET ratio response to different BAI-2 concentrations is used to generate the ligand binding saturation curve. For the determination of the binding affinity (K_{d}) and the binding stoichiometry n, a nonlinear regression analysis equation (Equation 4) is used as shown below:

$$R = {R}_{max} - [ \frac{ns ( {R}_{max} - {R}_{min} )}{( {K}_{d} + s )} ] \qquad Equation 4$$

where R is the observed CLPY FRET ratio response for different ligand concentrations (s) and Rₘₐₓ and Rₘᵢₙ represent the ligand-free and ligand-saturated FRET ratios, respectively.

BAI-2 Ligand Detection and Quantification from V. Harveyi Cultures:

For the detection of BAI-2 in V. harveyi cultures, BB120 (wild-type) and MM30 (luxS⁻) cells are used. V. harveyi is grown overnight at 28° C. for 16 hours and used to make a 2% (v/v) inoculum in fresh AB medium (2.0 mL, 0.5 mM borate) in round-bottom polystyrene tubes (BD Biosciences). The cell density is determined by plating serial dilutions of V. harveyi cultures on LM agar plates. Every 2.5 hours, the BB120 cell-free supernatant is collected by centrifugation at 4° C. for BAI-2 detection. To remove proteins (proteases) and cell debris, the supernatant is passed through a 3 kDa membrane cutoff filter (3K Microsep centrifugal device, Pall Life Science). For the determination of the unknown BAI-2 concentration, 200 μL of various dilutions of a 7.5 hours old V. harveyi BB120 (wild-type) culture supernatant grown in borate-free AB medium is added to CLPY (1.0 mL, 0.015 mg/mL) to generate a saturation curve. Cell-free supernatant from a 7.5 hours old V. harveyi MM32 culture is used as a control. Using a nonlinear regression relationship similar to Equation 4 (see Equation 5), the CLPY response to various dilutions of V. harveyi supernatant V is used in determining the half-saturation volume (h).

$$R = {R}_{max} - [ \frac{nv ( {R}_{max} - {R}_{min} )}{( h + v )} ] \qquad Equation 5$$

The unknown BAI-2 concentration M is determined using the relationship shown below.

M=K_{d}/h Equation 6

Bacterial Reporter Bioassay and EC₅₀ Determination:

V. harveyi strain MM30 (luxS⁻) is grown overnight at 28° C. for 16 hours and diluted 1:5000 in fresh borate-free AB medium. Bacteria (90 μL) are added to a 96-well microtiter plate containing 10 μL dilutions of the sample and incubated for 4 hours at 30° C. Luminescence is read using a Wallac Victor2 multimode plate reader (Perkin-Elmer). The BAI-2 concentration required for 50% maximum luminescence response (EC₅₀) is determined using a relationship based on the luminescence response L for different ligand concentrations (s)

$$L = {L}_{min} + [ \frac{s ( {L}_{max} - {L}_{min} )}{( {LC}_{50} + s )} ] \qquad Equation 7$$

where Lₘᵢₙ and Lₘₐₓ represent the minimum and maximum luminescence responses, respectively, at the time of measurement.

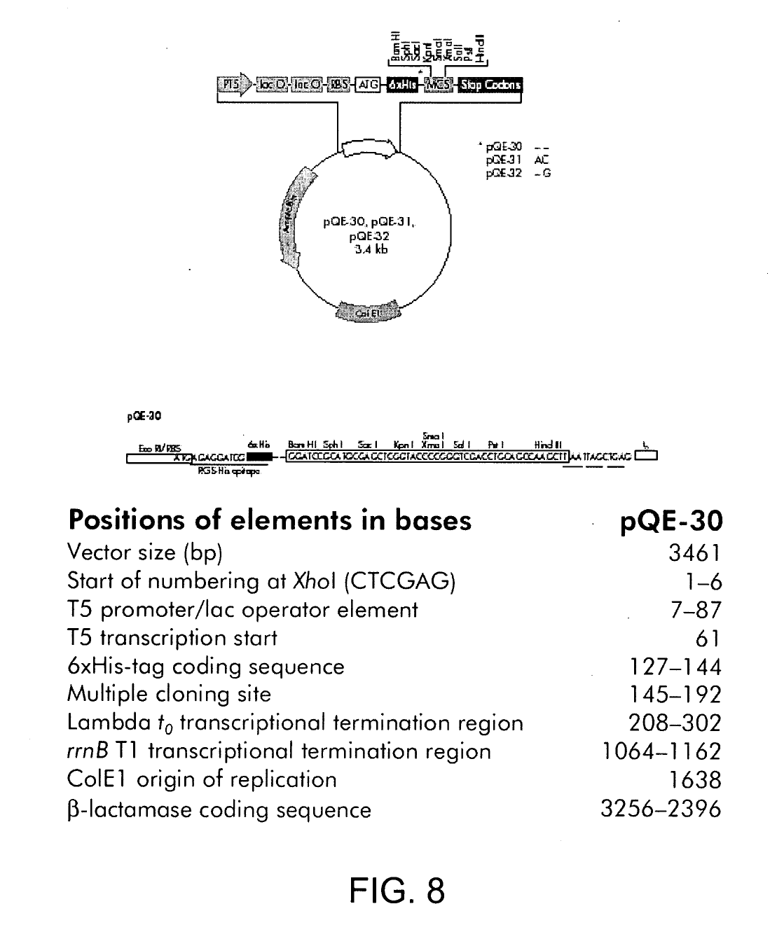

In one embodiment, the fusion protein of the present invention is made according to the following process. The BL21(luxS⁻) strain of E. coli is transformed with the a commercial vector carrying the CLPY gene. Any of a wide variety of appropriate vectors can be used; however, in this embodiment Qiagen's pQE-30 vector is selected. A diagram of this vector is shown in FIG. 8. Although the diagram does not include the specific location of the CLPY gene, it is located between the KpnI and PstI endonucleases sites in the multi-cloning site (labeled MCS in FIG. 8).

The cells are grown at 28° C. until the culture reaches about 0.6 AU at 600 nm. Then the cells are induced to overexpress CLPY by adding 0.3 mL of 1.0 M isopropyl-beta-D-thiogalactoside (IPTG) per 1.0 L of cell culture. Overexpression is allowed to continue for about six hours.

The fusion protein produced by the foregoing procedure can be purified according to the following procedure. Following overexpression, the cells carrying the pQE-30-CLPY vector are lysed by sonication and purified using the following buffer systems and Ni-NTA column:

Lysis buffer: 25 mM Sodium Phosphate pH 8.0, 35 mM Sodium Chloride, 10 mM Imidazole and 15 mM Beta-mercaptoethaonol;

Column buffer: 25 mM Sodium Phosphate pH 8.0, 35 mM Sodium Chloride, 10 mM Imidazole; and

Elution buffer: 25 mM Sodium Phosphate pH 8.0, 35 mM Sodium Chloride, 50 mM Imidazole.

### Results:

Characterization of the FRET-Based BAI-2 Biosensor:

The bPBPs typically have two globular protein domains tethered by a flexible hinge region that encompasses the ligand binding site. The two globular domains move about the hinge region in response to ligand binding. To monitor the binding of BAI-2 to LuxP, in one embodiment, a cyan fluorescent protein (CFP) and a yellow fluorescent protein (YFP) are fused to the surface-exposed N- and C-termini of the LuxP protein (FIG. 5(a)). In addition, a six-His tag is included at the N-terminus of the CLPY biosensor for rapid metal affinity purification. To ensure isolation of a ligand-free biosensor complex, the protein is expressed in an E. coli mutant (luxS⁻) which is unable to synthesize BAI-2. A mutation is also introduced (L221K) into the CFP and YFP genes to reduce the potential level of fluorophore dimerization and associated FRET artifacts. It is noted that maturation of YFP fluorescence yield required incubation for an additional 5 hours at 21 to 23° C. after its extraction and purification from E. coli due to its delayed chromophore maturation. The mature BAI-2 biosensor protein fusion is designated CLPY.

Addition of BAI-2 to CLPY resulted in a nonlinear decrease in the YFP:CFP fluorescence ratio up to the point of substrate saturation (FIG. 5(a) and FIG. 2). Previous studies have shown that binding of ligand to other bPBP proteins can increase or decrease the distance between the N- and C-termini, resulting in a decreased or increased FRET response, respectively. On the basis of the crystal structure model of ligand-complexed LuxP, the CFP and YFP domains of the BAI-2 biosensor are predicted to be sufficiently close (37 Angstroms) to facilitate efficient FRET. Therefore, a decrease in the FRET ratio following ligand binding implies that the fluorophores either move away from each other or substantially change their optical orientations. Analyses of the distances between the N- and C-termini of LuxP determined from the crystal structures of holoLuxP, with bound BAI-2, and apoLuxP, complexed with the periplasmic domain of LuxQ, indicate that there are no appreciable changes in the distance between the N- and C-termini of LuxP with or without bound BAI-2. Since FRET efficiency differs with respect to distance (r) by 1/r⁶, however, even small distance changes between the N- and C-termini may have large effects on FRET ratios.

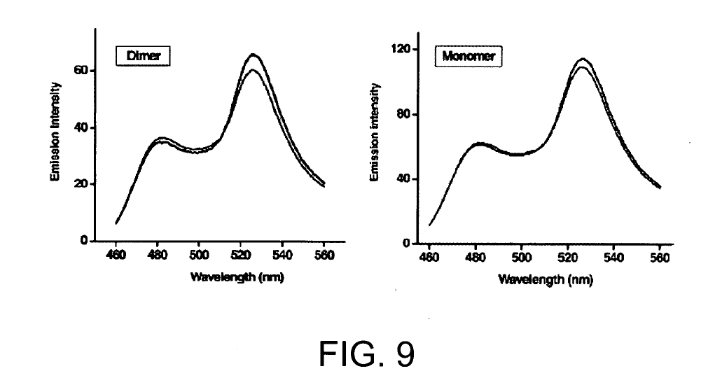

Changes in FRET ratios upon BAI-2 binding could also be attributed to intermolecular FRET due to end-to-end dimerization of CLPY. Furthermore, binding of ligand to LuxP has been shown to induce dissociation of LuxP dimers, potentially leading to a reduced level of intermolecular FRET. Therefore, a decrease in the level of FRET upon BAI-2 binding could be attributed to either a ligand binding-induced conformational change in LuxP (FIG. 5(a), Decreased FRET mechanism) or a shift from a dimeric to monomeric state. To determine if the loss of FRET is associated with a shift from a dimeric to monomeric state, the YFP:CFP fluorescence ratios of monomeric and dimeric CLPY separated by size exclusion chromatography are compared. Isolated CLPY is determined to be predominantly in a monomeric state (FIG. 7). Importantly, ligand-induced changes in the YFP:CFP fluorescence ratios are identical for monomeric (volumes of 106 to 114 mL) and dimeric (volumes of 91 to 99 mL) forms of CLPY (FIG. 9). These results suggest that intermolecular dimerization of CLPY does not contribute to the observed ligand binding-induced changes in FRET. With regard to FIG. 9, FRET responses of dimeric (91 to 99 mL) and monomeric (106 to 114 mL) CLPY to buffer (top lines) and 1.5 μM BAI-2 (bottom lines). The observed differences in the magnitude of the response between the dimeric and monomeric fractions (for added 1.5 μM BAI-2) are attributed to dissimilar protein concentrations in the representative samples.

CLPY Binding Affinity for BAI-2:

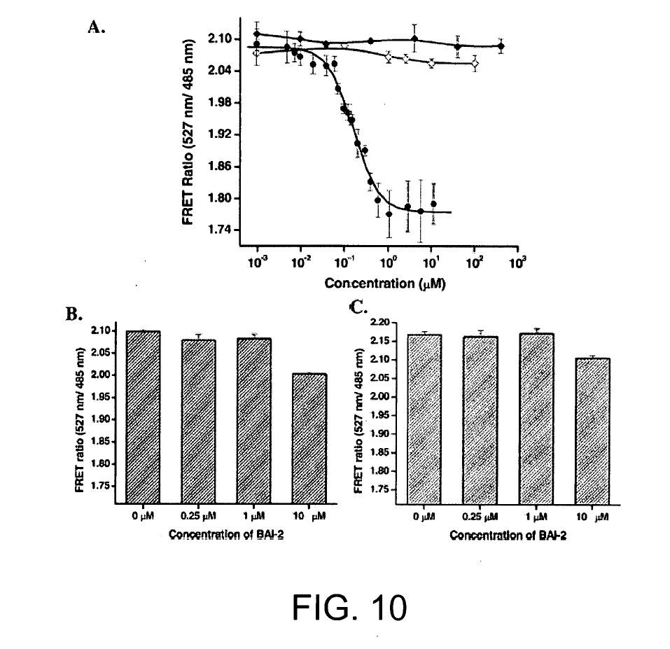

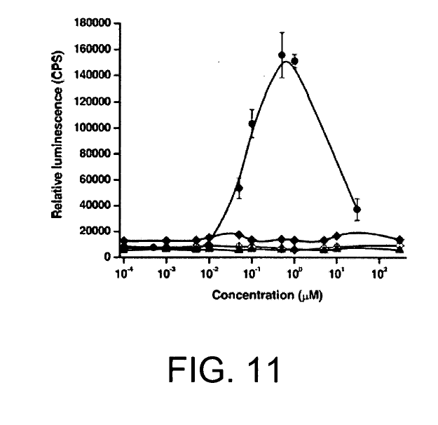

To determine the affinity of the CLPY biosensor for BAI-2, the protein is incubated with various concentrations of BAI-2 and the FRET response is measured. The maximum ligand-induced change in the YFP:CFP fluorescence emission ratio is approximately 0.32 (FIG. 10(a)). Nonlinear regression analysis of the concentration-dependent FRET response demonstrates that the apparent K_{d} for BAI-2 is 270 nM with a binding stoichiometry of 1.2 BAI-2 per molecule of CLPY. This high binding affinity is consistent with the ligand affinity constants measured for other bPBPs. To determine if the CLPY FRET response is specifically due to BAI-2 binding and not to interactions with structurally similar signaling molecules such as the AI-2 synthesis intermediate, DPD, FRET responses are measured in the presence of BAI-2 synthesis intermediates. CLPY did not exhibit any detectable FRET response to DPD (≦100 μM) or borate (≦400 μM) at high concentrations (FIG. 10(a)). To demonstrate that the BAI-2-dependent FRET response is due specifically to binding of BAI-2 to its LuxP binding site, a series of ligand binding site mutants are created. Site-specific mutations are introduced into the LuxP protein at several ligand binding site positions, including Q77A and S79A (designated M2CLPY) and Q77A, S79A, and W82F (designated M3CLPY). In the ligand-free state, both mature mutant biosensor proteins exhibited YFP:CFP ratios slightly higher than that of the wild-type protein. These FRET ratio increases may be attributed to the replacement of the glutamine and tryptophan side chains with smaller side chains such as alanine and phenylalanine, respectively. Following addition of BAI-2, both CLPY mutants had substantially reduced FRET responses compared to the wild-type CLPY protein. BAI-2 concentrations (≦1 μM) that yielded a maximum FRET response with the wild-type biosensor has little effect on the YFP:CFP ratio of the CLPY mutants. At supersaturating concentrations of BAI-2 (10 μM), the mutant biosensors M2CLPY and M3CLPY had only 30 and 18%, respectively, of the FRET response of the wild-type biosensor (FIGS. 10(b) and 10(c)). These results indicate that the BAI-2-dependent reductions in the CLPY YFP:CFP ratio are not associated with binding of BAI-2 to nonspecific binding site(s).

Comparison of CLPY Biosensor and Bacterial Reporter Bioassay Responses to BAI-2:

One potential advantage of bacterial bioassay systems of the present invention is potential amplification of the signal response following addition of the elicitor. To determine if the CLPY biosensor is as sensitive as the bacterial bioassay system for BAI-2, a comparison of the BAI-2 concentration-dependent response (luminescence) of the V. harveyi reporter strain, MM30, to the BAI-2-induced FRET response of the CLPY biosensor is made. In addition, a comparison of the relative responses of the two assay systems to DPD and borate are made. To prevent DPD from spontaneously forming BAI-2 in the presence of boron, V. harveyi MM30 cells are grown in borate-free AB medium. As shown in FIG. 11, the bacterial reporter strain exhibited a BAI-2 concentration-dependent response but did not respond to the addition of DPD or borate (FIG. 11). Using a nonlinear regression model, the EC₅₀ (50% effective luminescence yield concentration) bioluminescence response for BAI-2 is determined to be 100 nM. The V. harveyi bioluminescence response to BAI-2 is approximately 2.7 fold lower than the K_{d} (270 nM) of CLPY for BAI-2 (FIG. 10(a)). As observed in FIG. 11, however, the bioluminescence yield decreases with BAI-2 concentrations of greater than 0.5 μM. The reduction in bioluminescence at high BAI-2 concentrations demonstrates one of the limitations associated with in vivo bacterial bioassay systems. Unlike the bacterial system, the CLPY biosensor exhibited no reduction in FRET response in the presence of supersaturating (510 μM) concentrations of BAI-2 (FIG. 10(a)). These results imply that the reduction in bioluminescence in bacterial strains exposed to high concentrations of BAI-2 is not directly mediated by LuxP but may involve a feedback inhibition pathway or expression of a repressor that reduces the bioluminescence response.

Quantification of BAI-2 in Bacterial Culture Filtrates:

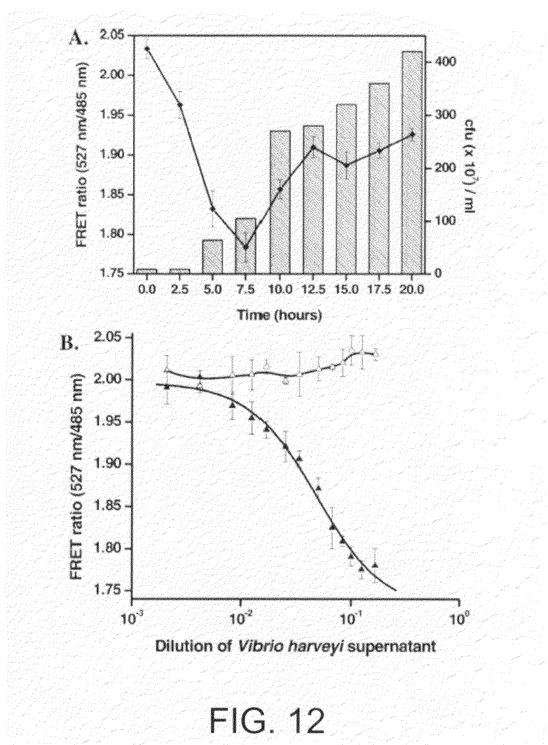

A CLPY biosensor is then used to determine BAI-2 levels in culture filtrates of wild-type V. harveyi (BB120) cells. Early to late log phase cultures exhibited a population-dependent increase in BAI-2 levels as indicated by a decrease in the YFP:CFP fluorescence intensity ratio (FIG. 12(a)).

With regard to FIGS. 12(a) and (b), these Figures are graphs illustrating the quantification of BAI-2 from V. harveyi cultures using the CLPY biosensor, where FIG. 12(a) is a graph illustrating wild-type V. harveyi BAI-2 levels monitored as a function of time and culture density. In the graph of FIG. 12(a) cell-free culture filtrates are prepared by passing the bacterial cell culture medium through a 3 kDa molecular mass cutoff filter. The filtrate (150 μL) is added to CLPY (0.015 mg/mL). The YFP:CFP FRET ratio (♦) is plotted as a function of cell density (×10⁷ cfu/mL; bars). The error bars represent the standard deviations from three independent experiments. Regarding FIG. 12(b), this graph represents a response of CLPY fluorescence to culture filtrate from wild-type (BB120) V. harveyi (▴) and LuxS⁻ mutant (MM32, Δ) mid- to late-log phase culture filtrate. The error bars represent the standard deviations from three independent experiments.

The highest BAI-2 levels are observed at a culture density of approximately 1×10⁹ cfu/mL (7.5 hours) (FIG. 12(a)). At this culture density, the experimentally determined BAI-2 concentration is 4.2 μM (FIG. 12(b)). Culture filtrates obtained from LuxS⁻ mutant (MM32) V. harveyi cultures that are unable to synthesize BAI-2 did not induce a change in the YFP:CFP ratio of the CLPY biosensor, indicating that the response is specific for BAI-2 and that other molecules in the growth media had no effect on the biosensor's BAI-2-dependent FRET response. Interestingly, as the culture aged, the BAI-2 levels decrease (10 to 20 hours, FIG. 12(a)). The results confirm the age-dependent decreases in BAI-2-mediated bacterial reporter responses observed earlier with V. harveyi cultures. An analogous age-dependent AI-2 production decrease has also been observed with organisms such as Vibrio Vulnificus, Salmonella typhimurium, and E. coli cultures. It is evident that V. harveyi actively synthesizes BAI-2 during the early logarithmic phase of culture growth, but at latter stages, BAI-2 levels drop, reflecting either an increased level of partitioning into the bacterial biomass or accelerated turnover.

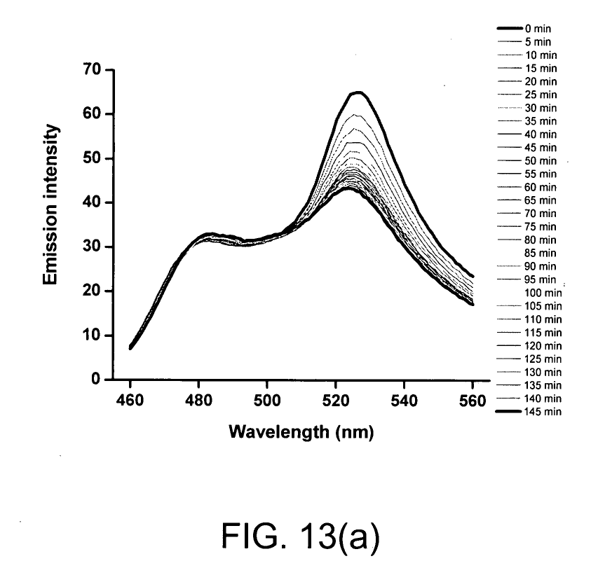

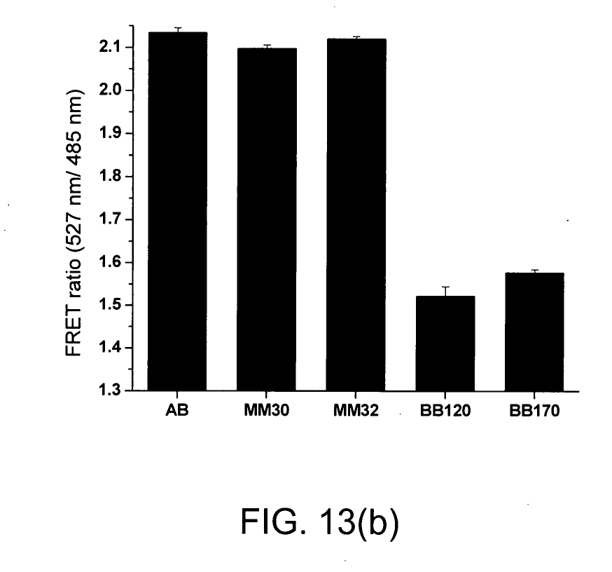

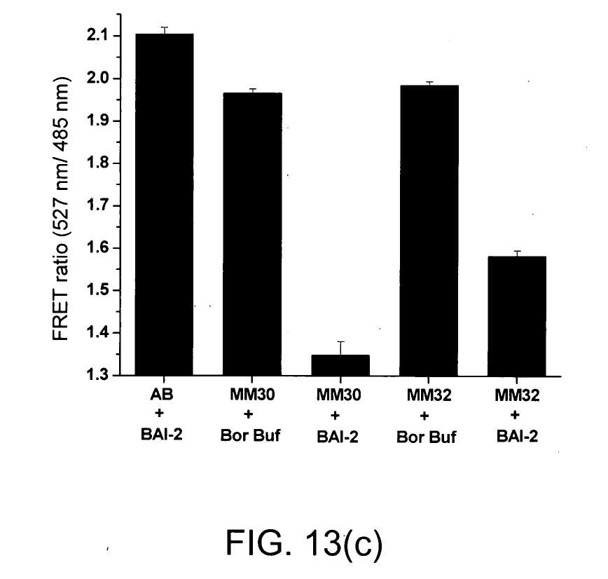

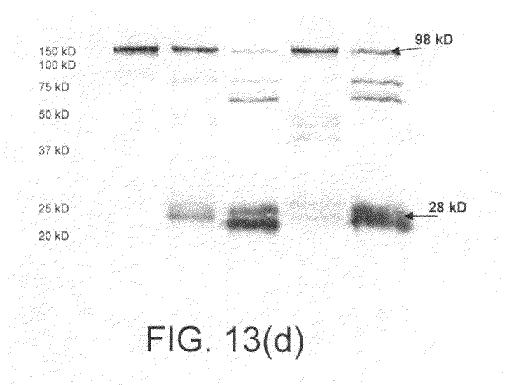

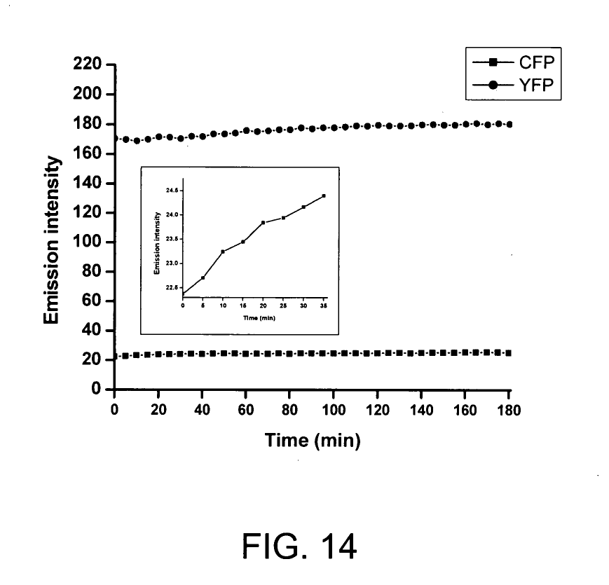

In one embodiment, the present invention comprises a bioassay directed to elucidating the effect of quorum sensing compounds on extracellular protease production. In this embodiment, a peptide or protein having a known sequence is fused to FRET donor/acceptor pairs as a means of causing reductions in FRET efficiency as a function of protease activity. More particularly, the present embodiment comprises the M3CLPY biosensing molecule, which fuses donor/acceptor pairs with a LuxP mutant protein that is insensitive to BAI-2 (FIG. 13). The LuxP moiety of M3CLPY is a relatively large protein (342 residues) with several endopeptidase cleavage sites (see Table 2).

TABLE 2

| | Number of |
|---|---|
| Name of Enzyme/Chemical | Cleavages |
| Arg-C Proteinase | 17 |
| Asp-N Endopeptidase | 27 |
| Asp-N Endopeptidase + N-Terminal Glu | 48 |
| BNPS-Skatole | 4 |
| CNBr | 5 |
| Chymotrypsin-High Specificity (C-Term to | 37 |
| [FYW], not before P) | |
| Chymotrypsin-Low Specificity (C-Term to | 76 |
| [FYWML], not before P) | |
| Clostripain | 17 |
| Glutamyl Endopeptidase | 21 |
| Hydroxylamine | 2 |
| Iodosobenzoic Acid | 4 |
| LysC | 22 |
| LysN | 22 |
| NTCB (2-Nitro-5-Thiocyanobenzoic Acid) | 1 |
| Pepsin (pH 1.3) | 106 |
| Pepsin (pH > 2) | 71 |
| Proline-Endopeptidase | 5 |
| Proteinase K | 158 |
| Staphylococcal Peptidase I | 21 |
| Thermolysin | 92 |
| Trypsin | 35 |

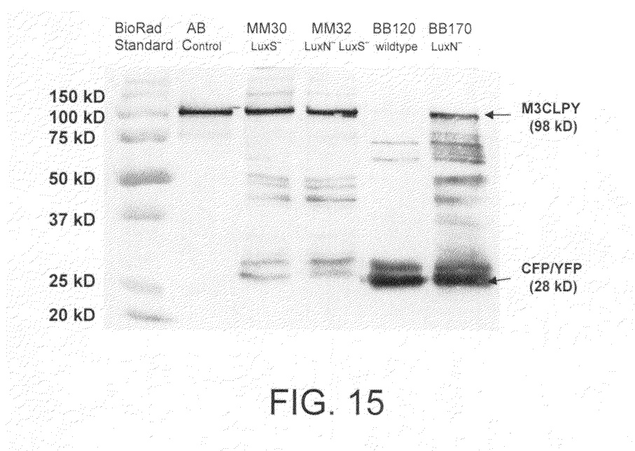

In order to establish whether or not the M3CLPY biosensor can be used as a reporter for extracellular protease activity, the biosensor is incubated with media containing one or more quorum sensing compounds. Specifically, cell-free culture filtrate is prepared from roughly 16 hours old V. harveyi strain BB120 culture, and incubated with M3CLPY. As shown in FIG. 13 and FIG. 15, M3CLPY exhibits a decrease in FRET efficiency in response to the cell-free filtrate.

Specifically, with regard to FIG. 15, FIG. 15 is a graph illustrating the monitoring of fluorophores in M3CLPY for fluorescence yields with V. harveyi BB120 cell-free culture filtrate addition. In the graphs of FIG. 15, mCFP (Ex440/Em485-slits 5 nm) and mYFP (Ex485/Em527-slits 5 nm) fluorescence are monitored for 3 hours using Cary Eclipse fluorometer (Varian inc). Given the disclosure contained herein, it is demonstrated that with the addition of BB120 cell-free filtrate the FRET efficiency is greatly reduced during this time frame. The mCFP and mYFP fluorescence data confirms the loss of FRET is not due to change in fluorophore intensity, but due to loss of energy transfer between mCFP and mYFP. Also in the inset is shown the increase in mCFP fluorescence intensity, due to reduced energy transfer of mCFP to acceptor mYFP.

Next the effect of various QS signal molecules on V. harveyi extracellular protease production is determined. Thus, various V. harveyi QS pathway mutant strains MM30 (luxS⁻), BB170 (luxN⁻), MM32 (luxS⁻, luxN⁻), and BB120 (wild type) are utilized. Cells grown in AB media for about 16 hours are used to make cell-free culture filtrate preparation for extracellular protease activity monitoring. Based on fluorescence measurements after 2.5 hours incubation of M3CLPY, FRET efficiency changes are evident (FIG. 13(b), FIG. 15). The BB120 and BB170 strains exhibit pronounced FRET decreases (notably, both strains have intact AI-2 mediated QS pathways). Accordingly, extracellular protease secretion increases with BAI-2 concentration.

This relationship is further supported by the following. Strains having the LuxS⁻ mutation (i.e., MM30 and MM32) are selected for extracellular protease recovery experiments. Fresh bacterial cultures (2% inoculum) in AB media are grown for about 4.5 hours (about 10⁹ cfu). Then 100 μM DPD with borate, or borate substituted buffer for control, is added and grown for additional 11.5 hours (about 5×10¹⁰ cfu). Cell-free culture filtrates are prepared from these strains and are monitored for extracellular protease recovery activity. M3CLPY FRET assays show that BAI-2 induces extracellular protease production. This result is confirmed with an immunoblot assay as shown in FIG. 13(c).

In another embodiment, the protein moiety for binding quorum sensing molecules comprises LsrB from Salmonella typhimurium, rather than LuxP. LsrB like LuxP also belongs to the bPBP family. Crystal structure analysis of both forms of LsrB shows limited change to the N- and C-termini distances. A donor/acceptor distance change of about 3.5 Angstroms resulting from ligand binding to LsrB should result in a FRET efficiency change of about 4.5%.

Currently, bacterial reporter bioassays are typically employed for the detection and quantification of QS signaling molecules. Bacterial reporter strains have also been used to detect compounds that interfere with QS and/or QS mimics produced by higher organisms. QS bioassay systems are, however, subject to substantial environmental perturbations. Previously, it was observed that the detection of AHL QS mimics produced by Chlamydomonas reinhardtii and detected using a V. harveyi reporter strain (BB170, luxN⁻) exhibited large disparities in reporter response between replicate experiments (unpublished data). Similar observations have been made by other groups using bacterial bioassay systems and are known to those of skill in the art. Fluctuations in culture pH and metabolite and growth inhibitor concentrations may all affect QS bioassays. These limitations clearly emphasize the need for caution when interpreting the results from different bioassay experiments.

In contrast to bacterial reporter assay systems, the CLPY biosensor undergoes a highly reproducible shift in the YFP: CFP fluorescence ratio upon ligand binding. This ligand-dependent reduction in the FRET ratio is associated with an intramolecular conformational change in the LuxP protein following ligand binding possibly involving a decrease in the distance between the N- and C-termini of LuxP (FIGS. 2, 7 and 9). Control experiments with BAI-2 precursors and LuxP BAI-2 binding site mutations demonstrate that the biosensor response is specific for BAI-2 (FIG. 10). For the first time, the present invention measures an apparent in vitro K_{d} for synthetic BAI-2 (270 nM) bound to LuxP. The affinity of LuxP for BAI-2 is consistent with expectations from bacterial reporter bioassay experiments.

Interestingly, analyses of BAI-2 concentrations in wild type V. harveyi cultures indicate that BAI-2 levels initially increase as the culture grows to mid-log phase and then decrease as the culture ages and its density increases (FIG. 12). These results suggest that rapid induction of QS-mediated gene expression may be necessary for effective regulation of QS-mediated responses. However, even at high culture densities, the effective BAI-2 concentration is sufficient to induce a QS response on the basis of the affinity of LuxP for BAI-2. Finally, the CLPY biosensor offers many advantages over current bioassays for the detection and quantification of BAI-2 QS compounds. The CLPY assay system is rapid (5 minutes) relative to conventional BAI-2 bioassays (3 to 6 hours) (FIG. 11), and it is not subject to a reduced signal response at high BAI-2 concentrations (FIG. 10(a)). These attributes are particularly useful for monitoring BAI-2 levels in bacterial cultures as well as in fluids obtained from infected tissues. In this latter capacity, BAI-2 biosensors can be used to monitor indirectly and non-invasively infection states in host organisms and the efficacy of treatments used to control those infections.

In one embodiment the biosensor of the present invention is used to monitor the state of an infection. In this embodiment, larger amounts of AI-2 generally infer the presence of larger amounts of infectious cells. Thus, the state of infection is monitored as a function of amount of infectious cells.

In another embodiment the present invention is used to monitor the level of quorum sensing compounds in various medical devices, including catheters. According to this embodiment, higher bacterial levels result in higher quorum sensing compounds levels, which can result in bacterial bio-film formation in the device and ultimately infection in the patient. Therefore, in this embodiment the present invention is used to detect the need for remedial measures, and/or check their effectiveness.

In a still further embodiment, the present invention is used to identify molecular mimics of quorum sensing compounds. This embodiment can be useful in drug discovery screening protocols for drug candidates. For instance, some pharmaceutically relevant mimics of quorum sensing compounds may bind with the biosensor of the present invention.

Although the invention has been described in detail with particular reference to certain embodiments detailed herein, other embodiments can achieve the same results. Variations and modifications of the present invention will be obvious to those skilled in the art and the present invention is intended to cover in the appended claims all such modifications and equivalents.

1. A fusion protein comprising SEQ ID 8.

2. A fusion protein comprising
a LuxP moiety, wherein the LuxP moiety is disposed between a donor and acceptor moiety;
a cyan fluorescent protein donor moiety connected to the LuxP moiety; and
a yellow fluorescent protein acceptor moiety connected to the LuxP moiety, wherein the yellow fluorescent protein is adapted to be substantially insensitive to chloride ion concentration, and wherein the donor and acceptor moieties disposed so that they are capable of fluorescent energy transfer when no ligand is bound to the LuxP moiety, and exhibit diminished or no fluorescent energy transfer when ligand bands to LuxP.

3. (canceled)

4. (canceled)

5. (canceled)

6. (canceled)

7. (canceled)


SEQUENCE LISTING (10 sequences)

SEQ ID NO: 1 - protein, 238 aa, Aequorea victoria
MSKGEELFTGVVPILVELDGDVNGHKFSVSGEGEGDATYGKLTLKFICTTGKLPVPWPTLVTTFCSYVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 2 - protein, 342 aa, Vibrio harveyi
VLNGYWGYQEFLDEFPEQRNLTNALSEAVRAQPVPLSKPTQRPIKISVVYPGQQVSDYWVRNIASFEKRLYKLNINYQLNQVFTRPNADIKQQSLSLMEALKSKSDYLIFTLDTTRHRKFVEHVLDSTDTKLILQNITTPVREWDKHQPFLYVGFDHAEGSRELATEFGKFFPKHTYYSVLYFSEGYISDVRGDTFIHQVNRDNNFELQSAYYTKATKQSGWDAAKASLAKHPDVDFIYACSTDVALGAVDALAELGREDIMINGWGGGSAELDAIQKGDLDITVMRMNDDTGIAMAEAIKWDLEDKPVPTVYSGDFEIVTKADSPERIEALKKRAFRYSDN

SEQ ID NO: 3 - protein, 342 aa, Vibrio harveyi; identical to SEQ ID NO: 2

SEQ ID NO: 4 - protein, 342 aa, Vibrio harveyi; identical to SEQ ID NO: 2

SEQ ID NO: 5 - protein, 157 aa, Bacillus subtilis
MPSVESFELDHNAVVAPYVRHCGVHKVGTDGVVNKFDIRFCQPNKQAMKPDTIHTLEHLLAFTIRSHAEKYDHFDIIDISPMGCQTGYYLVVSGEPTSAEIVDLLETDMKEAVEITEIPAANEKQCGQAKLHDLEGAKRLMRFWLSQDKEELLKVFG

SEQ ID NO: 6 - DNA, 5968 bp, Artificial Sequence
  Plasmid pQE-30
ctcgagaaatcataaaaaatttatttgctttgtgagcggataacaattataatagattcaattgtgagcggataacaatttcacacagaattcattaaagaggagaaattaactatgagaggatcgcatcaccatcaccatcacggatccgcatgcgagctcggtaccatgaagggcgaggagctgttcaccggtgtggtccccatcctggtggagctggacggcgacgtgaacggccacaagttctccgtctccggcgagggtgagggtgacgccacctacggcaagctgaccctgaagttcatctgcaccaccggcaagctgcccgtgccctggcccaccctggtcaccaccctgacctggggtgtgcagtgcttctcccgctaccccgaccacatgaagcagcacgacttcttcaagtccgccatgcccgagggctacgtgcaggagcgcaccatcttcttcaaggacgacggcaactacaagacccgcgccgaggtcaagttcgagggcgacaccctggtgaaccgcatcgagctgaagggcatcgacttcaaggaggacggcaacatcctgggccacaagctggagtacaactacatctcccacaacgtgtacatcaccgccgacaagcagaagaacggcatcaaggccaacttcaagatccgccacaacatcgaggacggctccgtgcagctggccgaccactaccagcagaacacccccatcggcgatggccccgtgctgctgcccgacaaccactacctgtccatccagtccgccctgtccaaggaccccaacgagaagcgcgaccacatggtcctgaaggagttcgtcaccgctgccggcatcacccacggcatggacgagctgtacacggccgacggtgttttgaatgggtactggggttatcaagagtttttggacgagtttcccgagcaacgaaatctgaccaatgctttatcagaagcagtacgagcacagccggtcccactgtctaaaccgacacaacgcccgattaaaatatcagtggtttacccaggacagcaagtttcagattactgggtaagaaatattgcatcattcgaaaaacgtttgtataagttgaatattaactaccaactgaaccaagtgtttactcgtccaaatgctgatatcaagcaacaaagcttgtcattaatggaagcgctcaagagcaaatcggattacttgattttcacgcttgatacgacaagacaccgtaaatttgttgagcacgttttggactcaacgaacaccaaattgatcttgcaaaatatcactacaccagtccgtgagtgggacaaacatcaaccgtttttatatgtcggatttgaccacgcagaaggcagtcgtgaattagcaacagaattcggaaagttcttcccaaaacacacatattacagtgtgctctacttttctgaaggttatattagcgatgtgagaggtgatacttttattcaccaagtaaaccgtgataataactttgagctacaatcagcgtattacacgaaggcaaccaagcaatccggctatgatgctgcgaaagcgagtttagcaaaacatccagatgttgattttatctatgcatgttcgaccgacgtagcattaggtgcagtagacgcactggctgagttgggacgtgaagatattatgatcaatggctggggtggaggctctgctgagttagacgctatccagaagggtgatttagacatcaccgtcatgcgtatgaatgatgacactggcatagccatggcagaagcgattaagtgggacttggaagataaaccagttccgaccgtatactcaggtgactttgaaatcgtaacaaaggcagattcaccggagagaatcgaagcgctgaaaaagcgcgcatttagatattcagataatggaggagcggccgccaagggcgaggagctgttcaccggtgtggtccccatcctggtggagctggacggcgacgtgaacggccacaagttctccgtctccggcgagggtgagggtgacgccacctacggcaagctgaccctgaagttcatctgcaccaccggcaagctgcccgtgccctggcccaccctggtcaccaccctgggctacggtctgaagtgcttcgcccgctaccccgaccacatgaagcagcacgacttcttcaagtccgccatgcccgagggctacgtgcaggagcgcaccatcttcttcaaggacgacggcaactacaagacccgcgccgaggtcaagttcgagggcgacaccctggtgaaccgcatcgagctgaagggcatcgacttcaaggaggacggcaacatcctgggccacaagctggagtacaactacaactcccacaacgtgtacatcatggccgacaagcagaagaacggcatcaaggtgaacttcaagatccgccacaacatcgaggacggctccgtgcagctggccgaccactaccagcagaacacccccatcggcgatggccccgtgctgctgcccgacaaccactacctgtcctaccagtccgccctgtccaaggaccccaacgagaagcgcgaccacatggtcctgaaggagttcgtcaccgctgccggcatcacccacggcatggacgagctgtacaagtaactcgaggtcgacggtatcgataagcttgatatcgaattcctgcagaagcttccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 7 - DNA, 2526 bp, Artificial Sequence
  Plasmid pQE-30
atgagaggatcgcatcaccatcaccatcacggatccgcatgcgagctcggtaccatgaagggcgaggagctgttcaccggtgtggtccccatcctggtggagctggacggcgacgtgaacggccacaagttctccgtctccggcgagggtgagggtgacgccacctacggcaagctgaccctgaagttcatctgcaccaccggcaagctgcccgtgccctggcccaccctggtcaccaccctgacctggggtgtgcagtgcttctcccgctaccccgaccacatgaagcagcacgacttcttcaagtccgccatgcccgagggctacgtgcaggagcgcaccatcttcttcaaggacgacggcaactacaagacccgcgccgaggtcaagttcgagggcgacaccctggtgaaccgcatcgagctgaagggcatcgacttcaaggaggacggcaacatcctgggccacaagctggagtacaactacatctcccacaacgtgtacatcaccgccgacaagcagaagaacggcatcaaggccaacttcaagatccgccacaacatcgaggacggctccgtgcagctggccgaccactaccagcagaacacccccatcggcgatggccccgtgctgctgcccgacaaccactacctgtccatccagtccgccctgtccaaggaccccaacgagaagcgcgaccacatggtcctgaaggagttcgtcaccgctgccggcatcacccacggcatggacgagctgtacacggccgacggtgttttgaatgggtactggggttatcaagagtttttggacgagtttcccgagcaacgaaatctgaccaatgctttatcagaagcagtacgagcacagccggtcccactgtctaaaccgacacaacgcccgattaaaatatcagtggtttacccaggacagcaagtttcagattactgggtaagaaatattgcatcattcgaaaaacgtttgtataagttgaatattaactaccaactgaaccaagtgtttactcgtccaaatgctgatatcaagcaacaaagcttgtcattaatggaagcgctcaagagcaaatcggattacttgattttcacgcttgatacgacaagacaccgtaaatttgttgagcacgttttggactcaacgaacaccaaattgatcttgcaaaatatcactacaccagtccgtgagtgggacaaacatcaaccgtttttatatgtcggatttgaccacgcagaaggcagtcgtgaattagcaacagaattcggaaagttcttcccaaaacacacatattacagtgtgctctacttttctgaaggttatattagcgatgtgagaggtgatacttttattcaccaagtaaaccgtgataataactttgagctacaatcagcgtattacacgaaggcaaccaagcaatccggctatgatgctgcgaaagcgagtttagcaaaacatccagatgttgattttatctatgcatgttcgaccgacgtagcattaggtgcagtagacgcactggctgagttgggacgtgaagatattatgatcaatggctggggtggaggctctgctgagttagacgctatccagaagggtgatttagacatcaccgtcatgcgtatgaatgatgacactggcatagccatggcagaagcgattaagtgggacttggaagataaaccagttccgaccgtatactcaggtgactttgaaatcgtaacaaaggcagattcaccggagagaatcgaagcgctgaaaaagcgcgcatttagatattcagataatggaggagcggccgccaagggcgaggagctgttcaccggtgtggtccccatcctggtggagctggacggcgacgtgaacggccacaagttctccgtctccggcgagggtgagggtgacgccacctacggcaagctgaccctgaagttcatctgcaccaccggcaagctgcccgtgccctggcccaccctggtcaccaccctgggctacggtctgaagtgcttcgcccgctaccccgaccacatgaagcagcacgacttcttcaagtccgccatgcccgagggctacgtgcaggagcgcaccatcttcttcaaggacgacggcaactacaagacccgcgccgaggtcaagttcgagggcgacaccctggtgaaccgcatcgagctgaagggcatcgacttcaaggaggacggcaacatcctgggccacaagctggagtacaactacaactcccacaacgtgtacatcatggccgacaagcagaagaacggcatcaaggtgaacttcaagatccgccacaacatcgaggacggctccgtgcagctggccgaccactaccagcagaacacccccatcggcgatggccccgtgctgctgcccgacaaccactacctgtcctaccagtccgccctgtccaaggaccccaacgagaagcgcgaccacatggtcctgaaggagttcgtcaccgctgccggcatcacccacggcatggacgagctgtacaagtaa

SEQ ID NO: 8 - protein, 841 aa, Artificial Sequence
  Plasmid pQE-30
MRGSHHHHHHGSACELGTMKGEELFTGVVPILVELDGDVNGHKFSVSGEGEGDATYGKLTLKFICTTGKLPVPWPTLVTTLTWGVQCFSRYPDHMKQHDFFKSAMPEGYVQQRTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSIQSALSKDPNEKRDHMVLKEFVTAAGITHGMDELYTADGVLNGYWGYQEFLDEFPEQRNLTNALSEAVRAQPVPLSKPTQRPIKISVVYPGQQVSDYWVRNIASFEKRLYKLNINYQLNQVFTRPNADIKQQSLSLMEALKSKSDYLIFTLDTTRHRKFVEHVLDSTNTKLILQNITTPVREWDKHQPFLYVGFDHAEGSRELATEFGKFFPKHTYYSVLYFSEGYISDVRGDTFIHQVNRDNNFELQSAYYTKATKQSGYDAAKASLAKHPDVDFIYACSTDVALGAVDALAELGREDIMINGWGGGSAELDAIQKGALDITVMRMNDDTGIAMAEAIKWDLEDKPVPTVYSGDFEIVTKADSPERIEALKKRAFRYSDNGGAAAKGEELFTGVVPILVELDGDVNGHKFSVSGEGEGDATYGKLTLYFICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLKEFVTAAGITHGMDELYK

SEQ ID NO: 9 - protein, 841 aa, Artificial Sequence; identical to SEQ ID NO: 8
  Plasmid pQE-30

SEQ ID NO: 10 - protein, 841 aa, Artificial Sequence; identical to SEQ ID NO: 8
  Plasmid pQE-30